(19) United States
(12) Patent Application Publication
(10) Pub. No.: US 2016/0129045 A1
(43) Pub. Date: May 12, 2016

(54) COMPOSITION FOR WOUND-HEALING COMPRISING ADULT STEM CELLS AND ELASTIN-LIKE POLYPEPTIDES

(71) Applicant: DAEGU GYEONGBUK INSTITUTE OF SCIENCE AND TECHNOLOGY, Daegu (KR)

(72) Inventors: Won Bae JEON, Daegu (KR); Seong Kyoon CHOI, Daegu (KR); Jin Kyu PARK, Daegu (KR)

(73) Assignee: DAEGU GYEONGBUK INSTITUTE OF SCIENCE AND TECHNOLOGY, Daegu (KR)

(21) Appl. No.: 14/925,991

(22) Filed: Oct. 29, 2015

(30) Foreign Application Priority Data
Oct. 29, 2014 (KR) 10-2014-0148664

(51) Int. Cl. A61K 35/35; A61K 38/39; A61K 47/48; A61K 9/00

(52) U.S. Cl. CPC A61K 35/35; A61K 9/0014; A61K 38/39; A61K 47/48246

(57) ABSTRACT

Provided is a composition for wound-healing containing adult stem cells and elastin-like polypeptides, and more specifically, to a composition for wound-healing capable of effectively treating skin wounds by simultaneously administering elastin-like polypeptides along with adult stem cells thereby increasing the viability of the adult stem cells transplanted on the wounds and promoting angiogenesis.

### INCORPORATION OF SEQUENCE LISTING

The Sequence Listing that is contained in the file named “sequence_listing_GBLO0-007.ST25.txt”, which is 1520 bytes in size (measured in Windows XP).

### CROSS-REFERENCE TO RELATED APPLICATIONS

This U.S. non-provisional patent application claims priority under 35 U.S.C. §119 of Korean Patent Application No. 10-2014-0148664, filed on Oct. 29, 2014, the entire contents of which are hereby incorporated by reference.

### BACKGROUND OF THE INVENTION

The present invention relates to a composition for wound-healing containing adult stem cells and elastin-like polypeptides, and more specifically, to a composition for wound-healing capable of effectively treating skin wounds by simultaneously administering elastin-like polypeptides along with adult stem cells thereby increasing the survival rate of the adult stem cells transplanted on the wounds and promoting angiogenesis.

Depending on whether or not there is a destruction of skin surfaces, wounds can be classified into an open wound, such as an incised wound, laceration, a penetrating wound, abrasion, etc., where skin or mucous membranes are injured and thus the tissues inside the skin are exposed to the air, and a closed wound where there is no breakage in the skin or mucous membranes, which can occur due to contusion or twisting by a dull weapon, shock, or by being pulled or bent. The process of wound-healing includes the initiation of proliferation of epidermal cells such as fibroblasts, vascular endothelial cells, and keratinocytes, by intracellular factors, migration of the cells to the wound area, granulation tissue formation, angiogenesis, and reepithelization, thereby achieving tissue regeneration.

In the process of wound recovery, upon the occurrence of the initial cut wound, anabolic and catabolic processes occur for about 6 to 8 weeks in a balanced manner, during which generally about 30% to 40% of recovery including normal skin tissues occur. As collagenous fibers progressively cause crosslinking, there is an increase in tensile strength, thereby forming a scar in the state of hyperemic projection. However, as time passes, the shape of the scar gradually returns to a state similar to skin. When there is a mutual imbalance in anabolic and catabolic steps during wound healing, collagen fails to decompose and becomes hardened, and thus the scar will remain in a projected state. This kind of tissue is classified as a hypertrophic scar or keloid.

During the wound-healing process, extracellular matrix factors, such as fibrinogen, collagen, and elastin, play a crucial role in migration of the cells to the wound area, granulation tissue formation, and angiogenesis, and in particular, fibronectin plays an important role in the repair of skin wounds. After a skin wound, fibronectin forms thrombosis and promotes the inflammatory cells into the wound area for the establishment of homeostasis. Fibronectin thrombosis also promotes the formation of fibroblasts, endothelial cells and keratinocytes, granulation tissue and epidermis (Greaves et al., J. Dermatol. Sci., 72:206, 2013; Eming et al., J. Invest. Dermatol., 127:514, 2007).

Recently, studies have been actively performed on tissue engineering approaches for using bone marrow- or fat tissue-derived totipotent cells or stem cells for tissue regeneration potency (Bi et al., J. Am. Soc. Nephrol., 18(9):2486, 2007; Wagatsuma A, Mol. Cell Biochem. 304(1-2):25, 2007; Song et al., Int. J. Impot. Res., 19(4):378, 2007). As is well known, totipotent or stem cells are generally obtained from bone marrow. However, due to the difficulty in obtaining bone marrow and the limitation of immune rejection response occurring when stem cells of other people are transplanted, fat tissues are being used as a substitute source for stem cells (Zuk et al., Tissue Eng., 7:211, 2001; Mizuno et al., Plast. Reconstr. Surg., 109:199, 2002; Zuk et al., Mol. Biol. Cel., 13:4279, 2002).

However, most of the adult stem cells which are transplanted into the wound become apoptosized due to a lack of oxygen and nutrients, in particular, due to the loss of cell-matrix interaction. To overcome such limitations, the researchers in the related art have studied the methods of wound-healing by using scaffolds such as acellular dermal matrix, polymer-based carriers, etc., wound dressings, and adipose stem cells, and discovered that such methods are advantageous for maintaining the viability of adipose stem cells and for wound-healing (Liu et al., Tissue Eng. Part A, 17:725,2011; Jiang et al., Biomaterials, 34:2501, 2013).

Meanwhile, it has been known that TGPG[VGRGD(VGVPG)₆]₂₀WPC multiblock biopolymer (REP), formed by repeated fusion of elastin valine-glycine-valine-proline-glycine (VGVPG) pentapeptides which is one of the elastin-like polypeptides (ELP), and arginine-glycine-aspartate (RGD) ligand, is effective for tissue regeneration (Jeon et al., J. Biomed. Mater Res. A. 97:152, 2011; Korean Patent No. 13500900). One of the advantages of REP is that, as a response to temperature change, solubilized Rep destroys coacervates (elastin) to become hydrophobic at or above a particular transition temperature (Tₜ). Although the tissue regeneration effect of REP was confirmed in the prior art of Korean Patent No. 13500900, only the sole effect of REP was confirmed, and thus the effects of REP on the increase in viability of adult stem cells and the promotion of wound-healing were not disclosed.

Accordingly, the present inventors, by endeavoring to find a method for improving the effect of adult stem cells on the wound-healing of skin, have studied the possible role of REP as a matrix, and through concurrent treatment with REP and adult stem cells, the present inventors have strengthened the viability of transplanted adult stem cells via cell adhesion and promoted migration of cells toward the wound area, thereby confirming the wound-healing effect, the wound-healing promotion effect, and the promotion effect of re-establishing the angiogenic network, and thus completing the present invention.

### SUMMARY OF THE INVENTION

In order to overcome the limitations described above, a first object of the present invention is to provide a composition for wound-healing or promoting wound-healing which includes a multiblock biopolymer (REP) that is established by repeated fusion between adult stem cells, elastin-like polypeptides, and ligands.

A second object of the present invention is to provide a method for wound-healing or promoting wound-healing which includes the adult stem cells and elastin-like polypeptides.

In order to resolve the first object of the present invention described above, the present invention provides a composition for wound-healing or promoting wound-healing which includes a multiblock biopolymer (REP) that is established by repeated fusion between adult stem cells, elastin-like polypeptides, and ligands.

In an exemplary embodiment of the present invention, the stem cell may be at least one type of mesenchymal stem cell, neural stem cell, or hematopoietic stem cell, selected from the group consisting of a fat-derived stem cell, a bone marrow-derived stem cell, and an umbilical cord-derived stem cell.

In another exemplary embodiment of the present invention, the elastin-like polypeptide may be an elastin valine-glycine-valine-proline-glycine (VGVPG) peptide (polypeptide).

In another exemplary embodiment of the present invention, the ligand may be arginine-glycine-aspartate (RGD) or arginine-glycine-aspartate-serine (RGDS).

In another exemplary embodiment of the present invention, the multiblock biopolymer may be TGPG[VGRGD(VGVPG)₆]ₙ WPC (wherein n=10, 12, 15, or 20).

In another exemplary embodiment of the present invention, the multiblock biopolymer may be [VGRGD(VGVPG)₆]ₙ (wherein n=10, 12, 15, or 20).

In another exemplary embodiment of the present invention, the composition may contain 25 μM to 100 μM of a multiblock biopolymer and 5×10⁵ to 5×10⁶ adult stem cells.

In order to solve the second object of the present invention, the present invention includes a method for wound-healing or promoting wound-healing using the composition for wound-healing or promoting wound-healing.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1A illustrates data from measurements of REP absorbance. FIG. 1B illustrates the level of aggregates in the state of coacervates, FIG. 1C illustrates the level of inverse phase transition of Fam-REP, and FIG. 1D illustrates the change in absorbance according to the Fam-REP wavelength;

FIG. 2 confirms characteristics of adipose stem cells (EGFP-ASC), which are isolated in Example 2 of the present invention, by illustrating data regarding the expression level of cluster of differentiation (CD) protein which is analyzed by using flow cytometry;

FIG. 3A illustrates data regarding the level of wound-closure in each group in Example 3 of the present invention, FIG. 3B illustrates rate of wound-closure in each group in Example 3 of the present invention, FIG. 3C illustrates observation of re-epithelialization in each group in Example 3 of the present invention and FIG. 3D illustrates rate of re-epithelialization in each group in Example 3 of the present invention, FIG. 3E illustrates result of Western blot analysis in each group in Example 3 of the present invention according to the expression level of α-SMA, and FIG. 3F illustrates expression ratio of α-SMA/β-actin in each group in Example 3 of the present invention;

FIG. 4A illustrates data regarding the expression amount of VEGF by ELISA assay in each group according to Example 3 of the present invention; FIG. 4B illustrates data regarding the expression amount of CD31 by ELISA assay in each group according to Example 3 of the present invention; FIG. 4C illustrates data regarding the expression amount of VWF by ELISA assay; FIG. 4D illustrates data from observation of CD31 expressed on a cross section of a tubular structure formed near the wound area; FIG. 4E illustrates simultaneously-expressing cells of EGFP and CD31;

FIG. 5A illustrates immunofluorescent analysis data regarding the expression amount of EGFP according to time after transplantation of EGFP-ASC and/or REP to the wound; FIG. 5B and FIG. 5C illustrates Western blot analysis data regarding the expression amount of EGFP according to time after transplantation of EGFP-ASC and/or REP to the wound;

FIG. 6A and FIG. 6B illustrate data regarding the adhesion rate of ASC in REP, collagen I, collagen IV, and fibronectin measured according to time; FIGS. 6C-6J illustrate data regarding the activity level of phosphorylating Fak, Src, Erk, and Akt, measured by Western blot analysis.

FIG. 7A and FIG. 7B illustrate the ASC activity of phosphorylation of Erk through REP during wound-healing measured by using Western blot analysis; FIGS. 7C and 7D illustrate the ASC activity of phosphorylation of Akt through REP during wound-healing measured by using Western blot analysis.

### DETAILED DESCRIPTION OF PREFERRED EMBODIMENTS

The present invention will be explained in greater detail herein below.

As described above, the adult stem cells transplanted to the wound have a problem in that most of them become apoptosized due to lack of oxygen and nutrients, in particular, due to the loss of cell-matrix interaction.

In order to solve the problems described above, the present invention provides a composition for promoting wound-healing or wound-healing promotion containing multiblock biopolymer (REP) formed by repeated fusion of adult stem cells, elastin-like polypeptides, and ligands. Therethrough, there is an effect of not only increasing the viability of transplanted adult stem cells, but also of more effectively healing skin wounds through promotion of the introduction of cells to the wound area and of the re-establishment of angiogenic network.

The present invention provides a composition for promoting wound-healing or wound-healing promotion which includes adult stem cells, and a multiblock biopolymer (REP) formed by repeated fusion of elastin-like polypeptides and ligands.

As used herein, the term “stem cell” refers to a cell which serves as a basis of a cell or tissue, and may refer to an undifferentiated cell having the ability to be differentiated into a particular or a plurality of functional cells, and having a self-replication ability of being capable of repeatedly producing identical cells by oneself. Stem cells can be divided into embryonic stem cells (EScell) and adult stem cells, according to differentiation potency.

Adult stem cells are stem cells which are obtained in adults that have completed development or in the placenta during the developmental stage when body organs of an embryo are developed during embryogenesis, and the differentiation potency may generally be limited to those cells which constitute the tissues (multipotent). These adult stem cells remain in most body organs even after becoming an adult and serve to supplement the loss of cells which may occur normally or pathologically. Representative examples of adult stem cells may include hematopoietic stem cells present in bone marrow and mesenchymal stem cells which are differentiated into the cells of connective tissues other than blood cells. The hematopoietic stem cells may be differentiated into various blood cells including red blood cells, white blood cells, etc., and the mesenchymal stem cells may be differentiated into osteoblasts, chondroblasts, adipocytes, myoblasts, etc. The mesenchymal stem cells may be isolated from bone marrow, which is an important storage for mesenchymal stem cells, but there may be a difficulty in isolation thereof, and may also be isolated and cultured in fat tissues, etc. In the present invention, the mesenchymal stem cells may be all kinds of cells having stem cell potency, that is, differentiation potency and proliferation potency.

In the present invention, the adult stem cell may be at least one type of mesenchymal stem cell, neural stem cell, or hematopoietic stem cell, selected from the group consisting of anadipose-derived stem cell, a bone marrow-derived stem cell, and an umbilical cord-derived stem cell, and in the present invention, a fat-derived stem cell is desirably used. However, it should be noted that the generally used fat-derived stem cell was used in the present invention to confirm the excellent effect of the composition of the present invention for wound-healing or promotion of wound-healing, and other adult stem cells showing the effect of wound-healing may be used without limitations.

As the adult stem cells, fat-derived stem cells may be desirably used which are obtained by using fat tissues which are discarded during a frequently conducted liposuction procedure, and thus do not require an invasive procedure. The fat-derived stem cell may be obtained from mammals including humans, and preferably from human fat tissues or fat cells through procedures such as liposuction and sinking, enzyme treatment of collagenase, etc., and removal of suspension cells, such as red blood cells, etc., through centrifugation, in accordance with known methods which are disclosed in International Publication Nos. WO2000/53795 and WO2005/042730. The fat tissues may include brown or white tissues derived from subcutaneous, reticular membrane, intestines, breast germline, or other fat tissue regions, and can be easily obtained from the conventional liposuction procedure.

As used herein, the term “wound or a cut-wound” refers to an injured state of a body of a living organism, and includes a pathological state in which those tissues which establish the internal or external surfaces of a living organism, e.g., skin, muscles, neural tissues, bones, soft tissues, internal organs, or vascular tissues are cut out or destroyed. Examples of the cut-wound may include, but are not limited to, non-healing traumatic cut-wound, destruction of tissues by exposure to radiation, abrasion, osteonecrosis, laceration, avulsion, penetrated wound, gunshot wound, incised wound, burns, frostbite, contusion or bruise, skin ulcer, xeroderma, skin keratosis, cracks, burst, dermatitis, pain due to dermatophytosis, surgery wound, vascular disease wound, cut-wound such as corneal wound, pressure sore, decubitus, state related to diabetes such as diabetic skin erosion and circulation disorder, chronic ulcer, suture areas after plastic surgery, spinal cord injury wound, gynecological wound, chemical wound, eczema, etc., and an injury to any part of a subject.

In the present invention, the elastin-like polypeptide may be elastin valine-glycine-valine-proline-glycine (VGVPG) polypeptide and the ligand may be arginine-glycine-aspartate (RGD) or arginine-glycine-aspartate-serine (RGDS).

That is, the multiblock biopolymer (hereinafter, REP) is one established by repeated fusion of VGVPG peptide and RGD or RGDS, preferably TGPG[VGRGD(VGVPG)₆]ₙWPC (wherein=10, 12, 15, or 20), and more preferably, [VGRGD(VGVPG)₆]ₙ (where n=10, 12, 15, or 20).

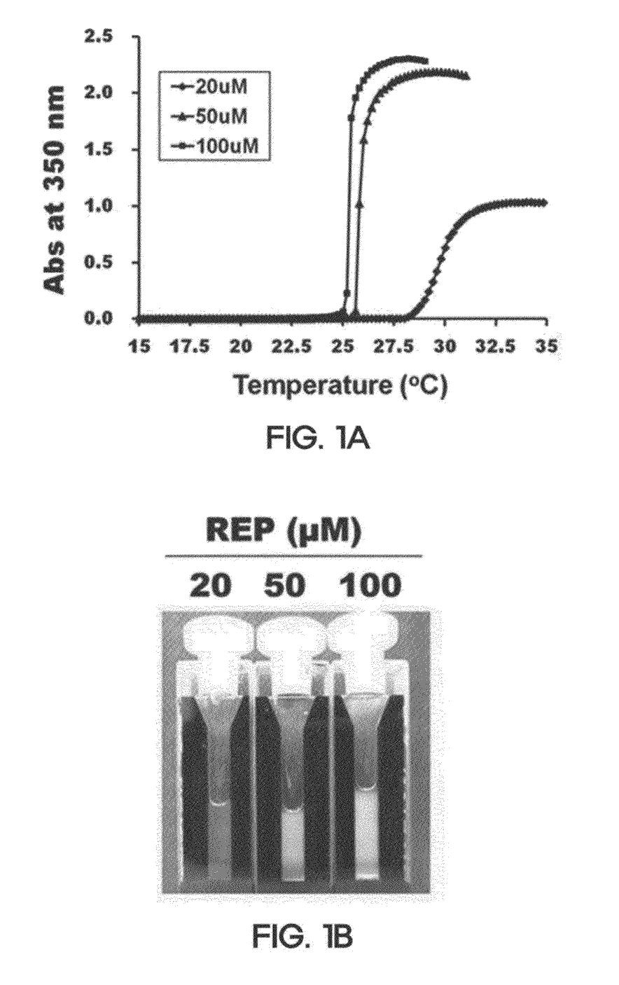

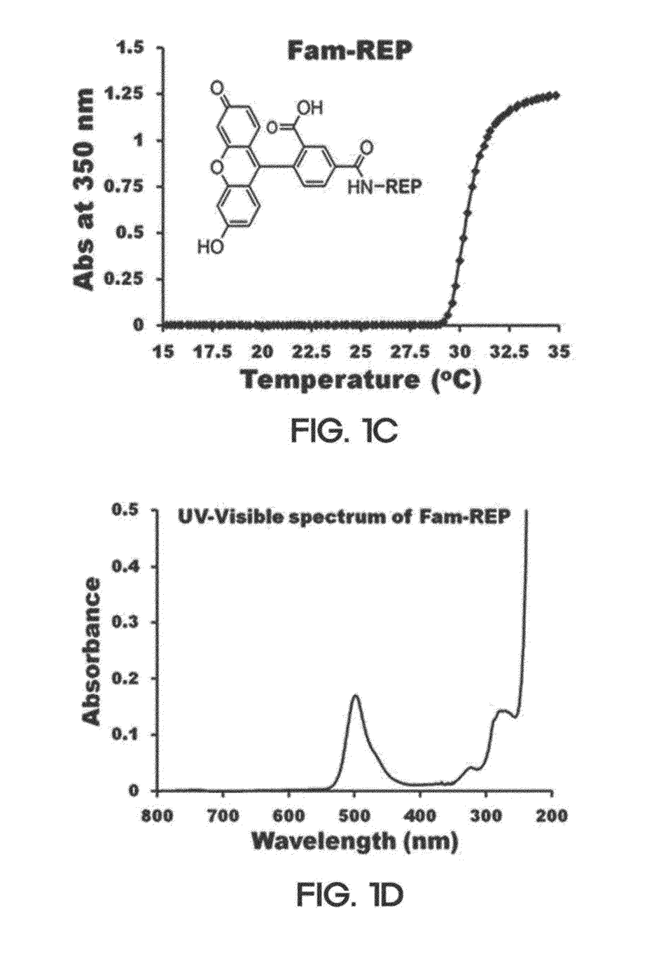

In an exemplary embodiment, REP was prepared through a known method (Jeon W B et al., J. Biomed. Mater. Res. A, 97:152, 2011), and its characteristics were confirmed by preparing Fam-labeled REP (Fam-REP). When the level of inverse phase transition of REP was measured in the presence of DTT, it was observed that absorbance was rapidly increased at 25° C. or higher (FIG. 1A), and the level of REP aggregation according to concentration was confirmed at 35° C. in the state of coacervates (FIG. 1B). Additionally, as shown in FIGS. 1C and 1D, Fam-REP showed an increase in absorbance at 30° C. and higher, and a peak around 500 nm. That is, since particular transition temperatures (Tₜ) of REP and Fam-REP are lower than the body temperature of mice, agglutination in the state of coacervates is possible within the wound.

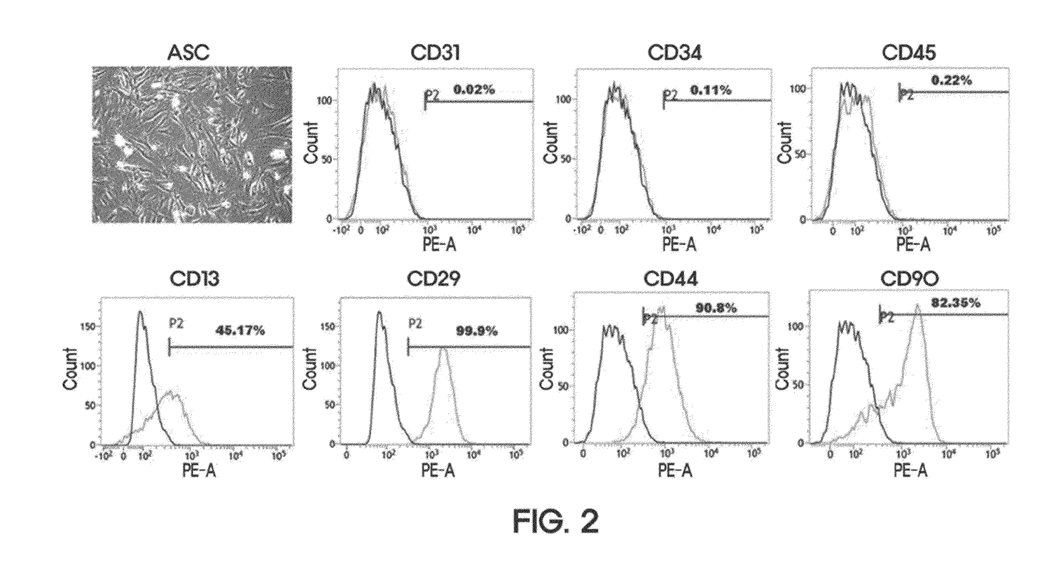

Additionally, in an exemplary embodiment of the present invention, for easy measurement of the state of adipose-derived stem cells which are transplanted in the wound, a labeled adipose stem cell (hereinafter, “ASC”) was isolated from a C57BL/6-GFP mouse, and then characteristics of ASC were analyzed by using flow cytometry. As a result, the cluster of differentiation markers for CD13, CD29, CD44, and CD90 were shown to be positive, and CD31, CD34, and CD45 were observed to be negative (FIG. 2).

In the present invention, the composition for wound-healing or promotion of wound-healing may contain a multiblock biopolymer at a concentration ranging from 25 μM to 100 μM and number of adult stem cells ranging from 5×10⁵ to 5×10⁶, and preferably, a multiblock biopolymer at a concentration ranging from 45 μM to 65 μM and 1×1⁶ adult stem cells. When the composition contains a concentration of the multiblock biopolymer lower than the above concentration and number of the adult stem cells lower than the above, the wound-healing ability may be decreased. Although a higher concentration of the multiblock biopolymer and a higher number of adult stem cells than the above range may be used, sufficient wound-healing or promotion of wound-healing effects may be realized, even from the above ranges. Additionally, when the concentration of the multiblock biopolymer is 100 μM or higher, a superior concentration dependent effect is not exhibited, and since an additional condensation process is required for establishing a concentration equal to or higher than 100 μM, there is a limitation in not being desirable from the perspective of cost. It was also confirmed that even a higher number of cells than the above range does not significantly increase the transplantation effect of the adult stem cells in a concentration dependent manner.

In an exemplary embodiment of the present invention, to confirm the effect of simultaneous administration of ASC and REP, each mouse was placed into either a Sham control group, a REP-treated group, an ASC-treated group, or an ASC-REP combined treatment group (RA), and administered to for wound-healing, and the synergy effects in the level of wound closure, restoration effect of local vascular structure, etc., according to time progress were examined in the ASC-REP combined treatment group.

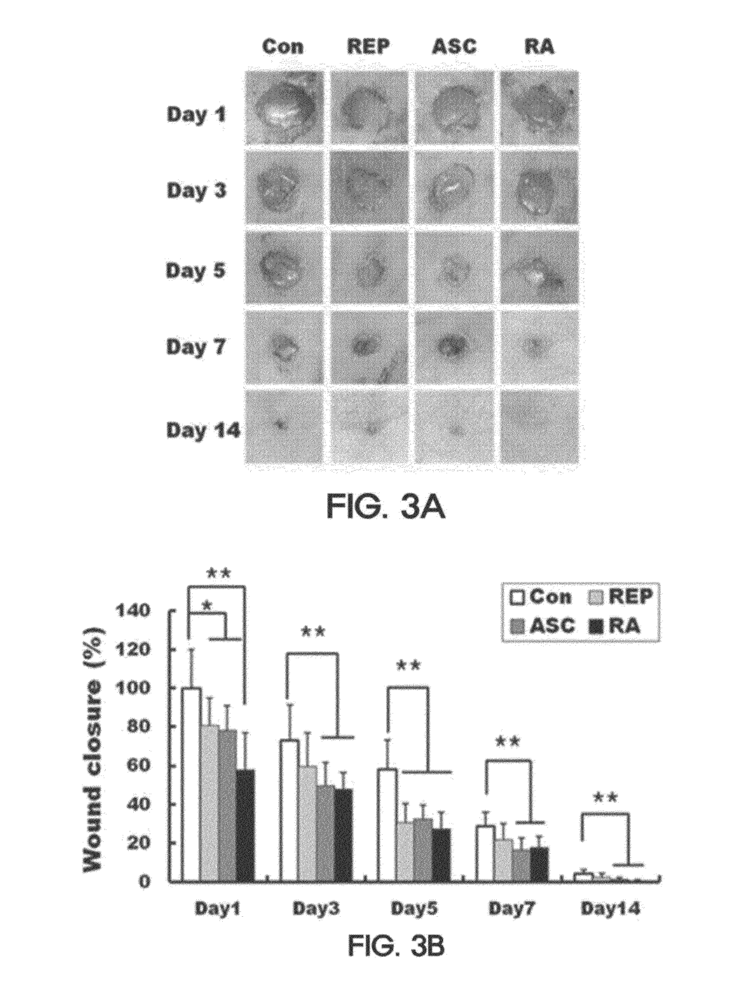

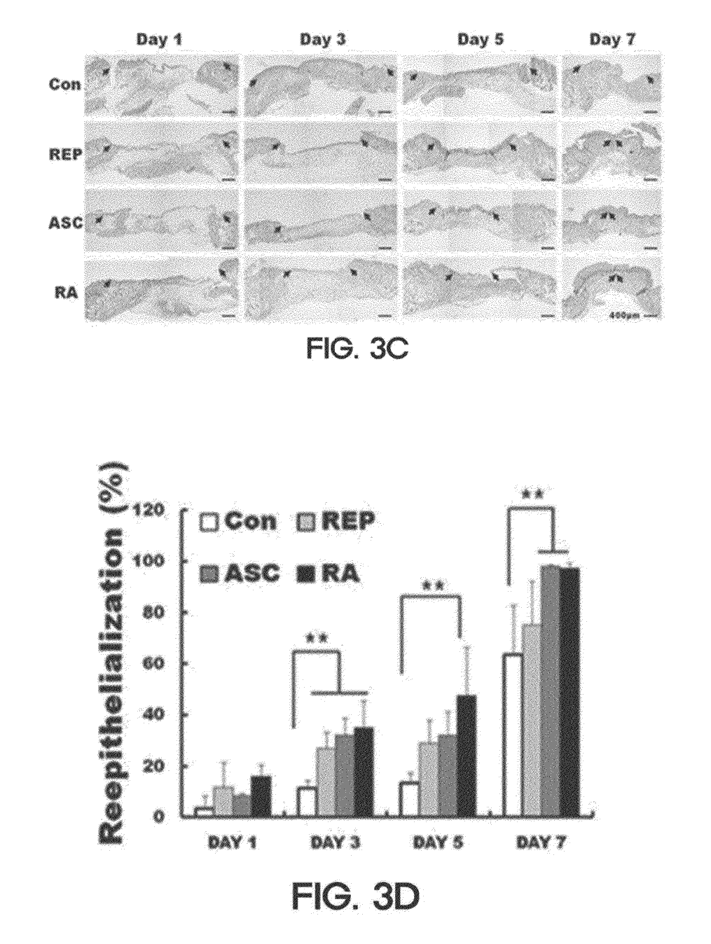

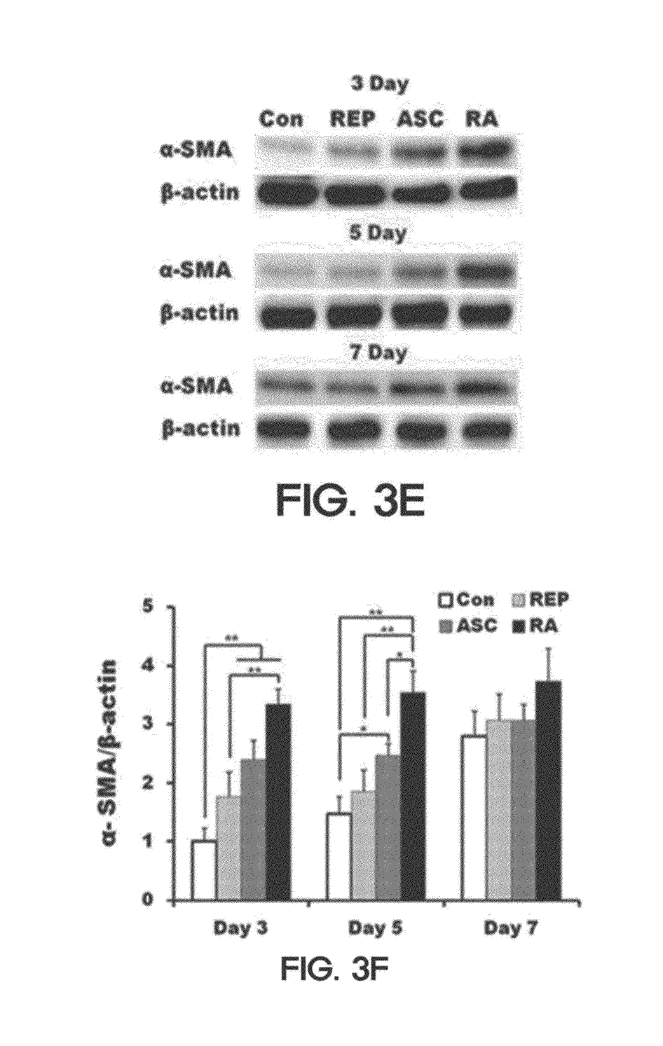

First, FIG. 3 confirms the level of wound-healing in each group according to Example 3 of the present invention. As shown in FIGS. 3A and 3B, all of REP-, ASC-, and ASC-REP-treated groups were confirmed to have more wound closure compared to the Sham Control, and the rate of wound closure was shown to increase in the order of REP, ASC, and RA(REP+ASC) treatment. Additionally, the same as in the above result, a high re-epithelialization was observed in RA group compared to other groups (FIGS. 3C and 3D). Regarding the α-SMA expression, as shown in FIGS. 3E and 3F, RA group showed a 1.4-, 1.4-, and 1.2-fold increase in α-SMA expression compared with the ASC treatment alone, in the 3^{rd}, 5^{th}, and 7^{th} day of the experiment, respectively.

That is, RA group showed an improvement of wound-healing compared with the single treatment of ASC or REP, and this indicates that the combined treatment of ASC and REP maximizes the wound-healing efficiency.

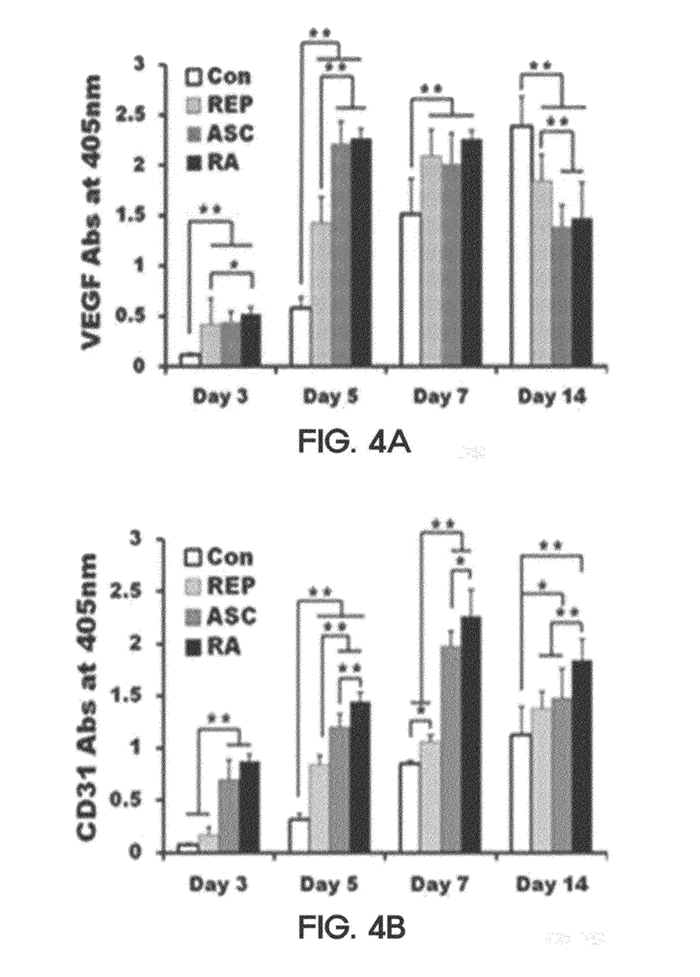

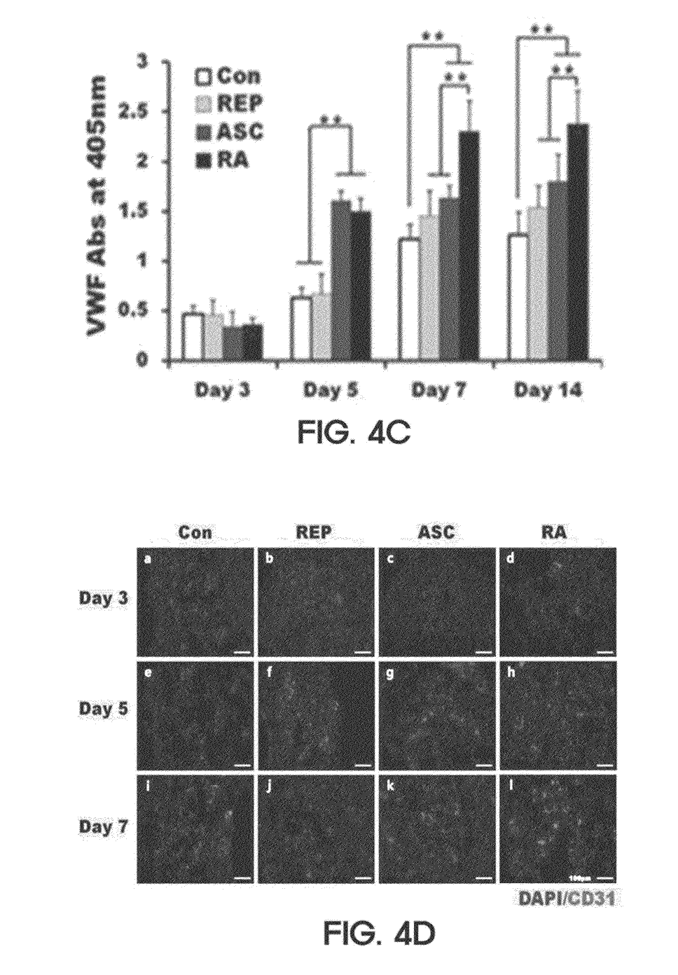

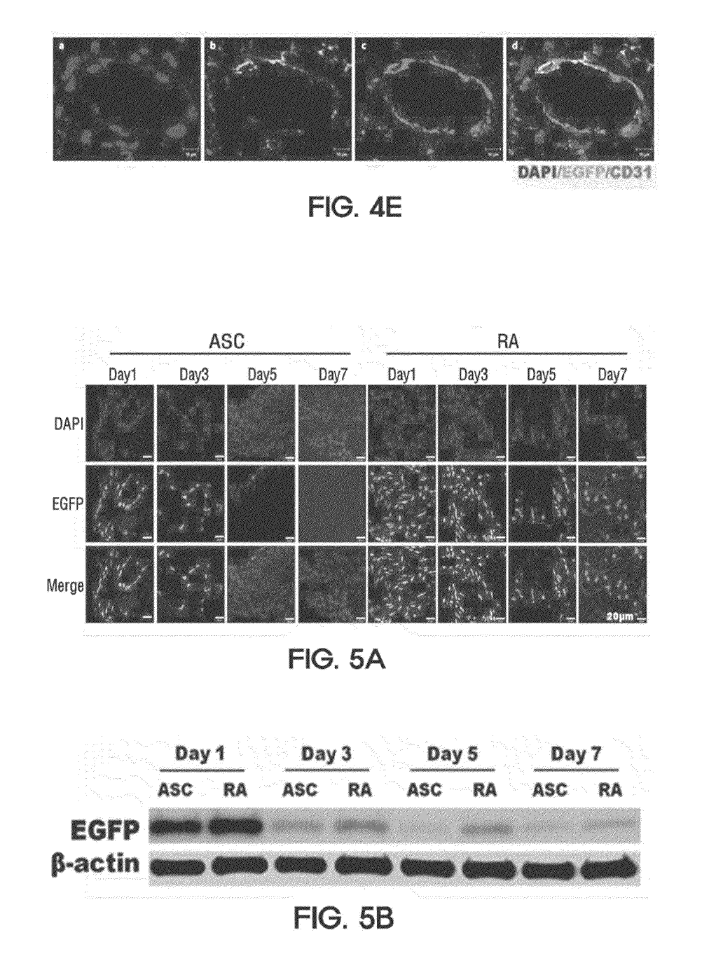

FIG. 4 shows the restoration effect of local vascular structures in each group according to Example 3 of the present invention. As shown in FIGS. 4A to 4C, in the case of the combined treatment of ASC and REP, the amount of production of VEGF, which is an angiogenesis-related factor, CD31, an indicator of epithelial layer, and VWF, which induces hemostasis by attaching platelets to the area with vascular injury was significantly higher than other groups, and the synergy effect of wound-healing by the combined treatment of ASC and REP was confirmed.

Additionally, when the expression level of CD31, which is expressed in the cross section of a tubular structure formed in wound areas, was measured, the group simultaneously treated with REP and ASC showed a high amount of CD31 expression (FIG. 4D), and this indicates that the group simultaneously treated with REP and ASC promoted the formation of microvascular structure in all of the steps.

As shown in FIG. 4E, when REP and ASC were simultaneously treated, cells which simultaneously express CD31 and EGFP along the cross section of tubular structure could be confirmed. That is, the discovery of cells which showed positive in both CD31 and EGFP along the cross section of newly formed vascular tubules indicates that the transplanted ASC are directly involved in the differentiation into the phenotype of endothelial cells and angiogenesis of regeneration of angiogenesis.

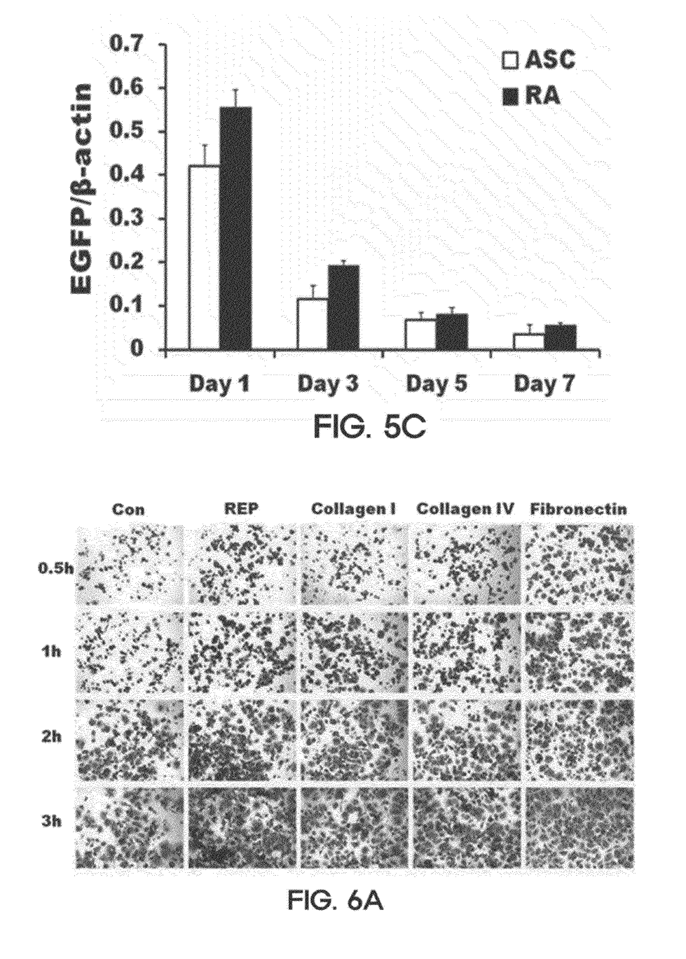

FIG. 5 confirms the effect of REP on the increase in viability of transplanted adipose stem cells. As shown in FIG. 5A, ASC (hereinafter, “EGFP-ASC”), which expresses EGFP, was detected in a higher amount in RA group than in the group treated with ASC alone. When EGFP, which is expressed in ASC, was analyzed by Western blot method, as shown in FIGS. 5B and 5C, the cell viability was increased due to the mixture of REP and ASC, by 24%, 40%, 17%, and 35%, on the 1^{st}, 3^{rd}, 5^{th}, and 7^{th} day of experiment, respectively.

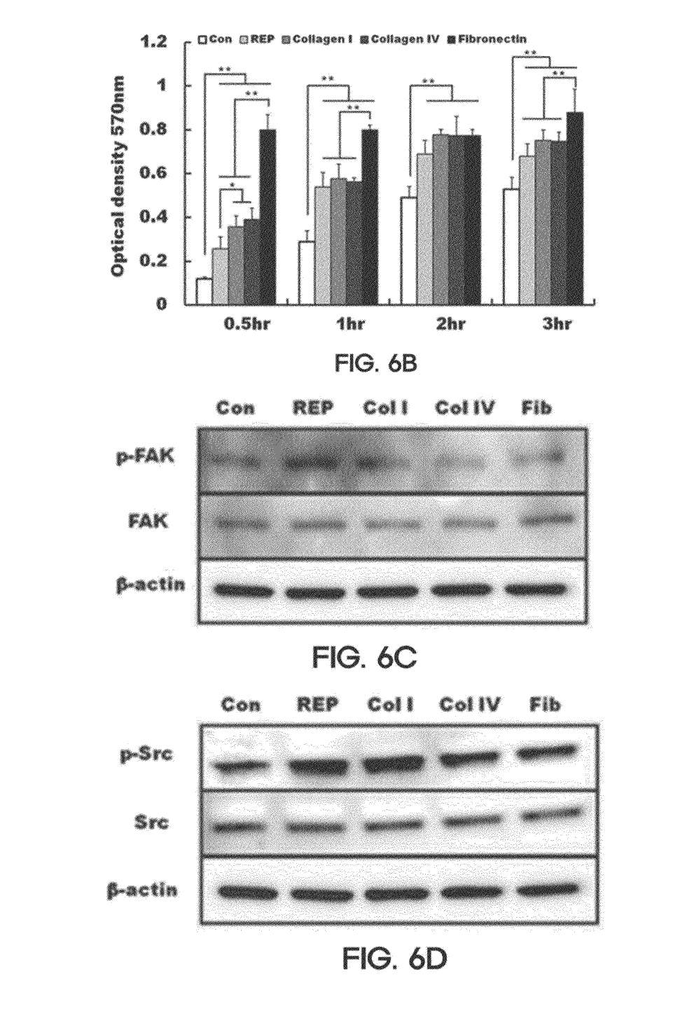

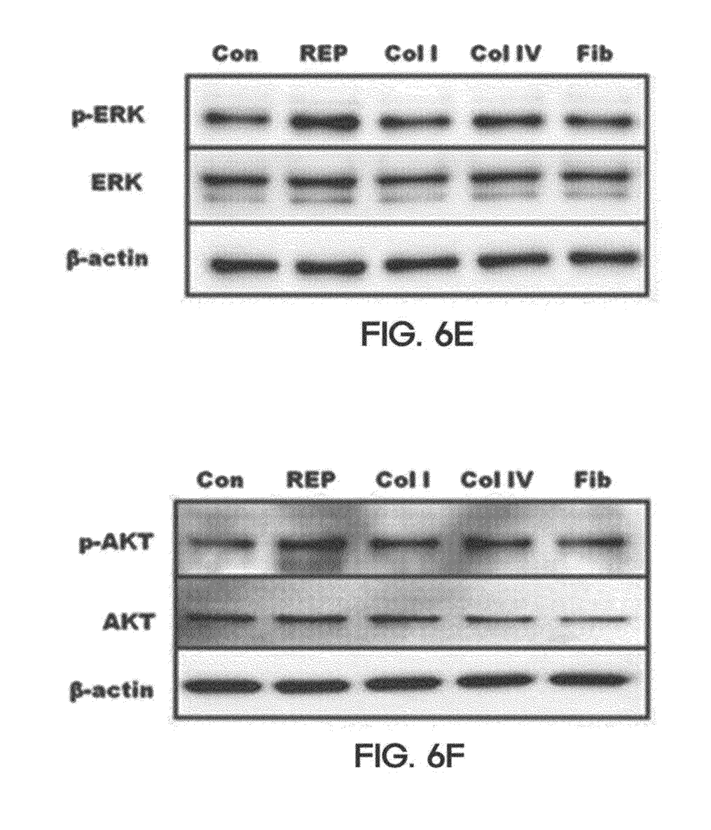

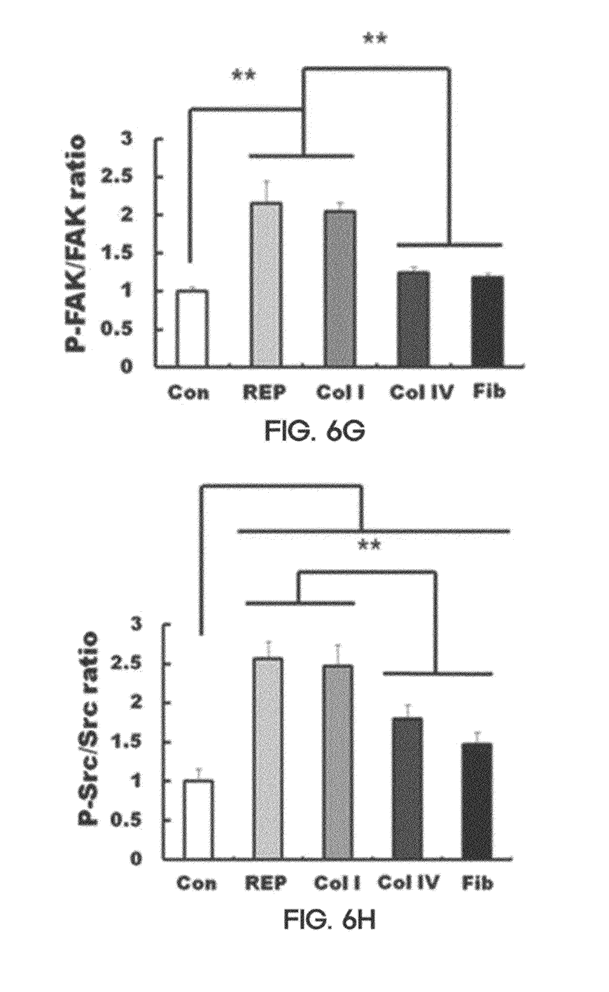

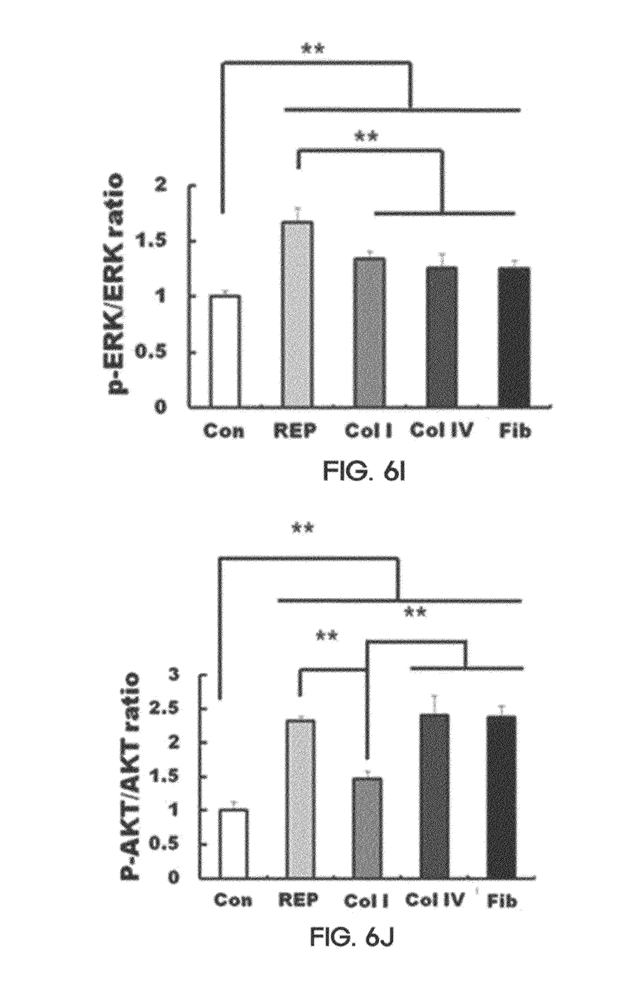

FIG. 6 confirms the ASC adhesion capacity and the activity of phosphorylation of focal adhesion kinase (Fak), Src (SRC proto-oncogene, non-receptor tyrosine kinase), extracellular-signal-regulated kinases (Erk), and protein kinase B (Akt) in REP. As shown in FIGS. 6A and 6B, the highest cell adhesion rate was shown in fibronectin rather than in REP. However, regarding the activation of phosphorylation of Fak, Src, Erk, and Akt, as shown in FIGS. 6C to 6J, the induction of activation of Fak, Src, Erk, and Akt (phosphorylation) was shown to significantly increase when ASC was cultured in REP, compared with other scaffolds.

Erk pathway is known to be associated with the increase of secretion of angiogenesis factors including VEGF from ASC and keratinocytes. Additionally, the activation of Akt signal is known to increase the production of VEGF in keratinocytes and promote collagen complexes, neovascules, and the maturation of blood vessels. Additionally, the increase in activity of Fak and Src indicates the promotion of regeneration of epithelial cells in epidermis, dermal layer, and vascular endothelial cells, and confirms that such is related to the rapid migration of endogenous cells from the wound area as migrating signal is induced through RGD of REP.

That is, it is speculated that REP not only serves to induce the phosphorylation of Erk and Akt in ASC cells, thereby promoting angiogenesis, but also increases the activity of Fak and Src, thereby contributing to the migration of endothelial cells to the wound.

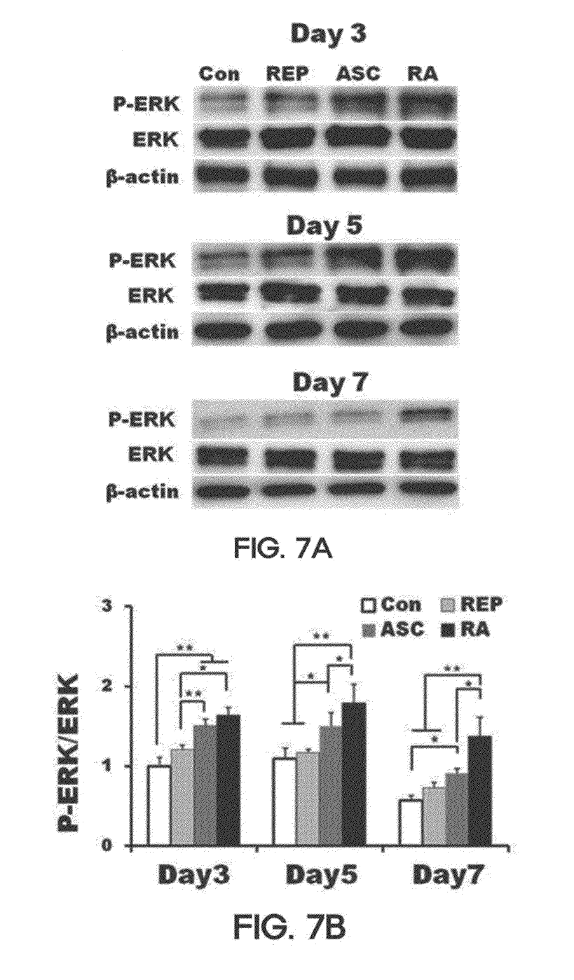

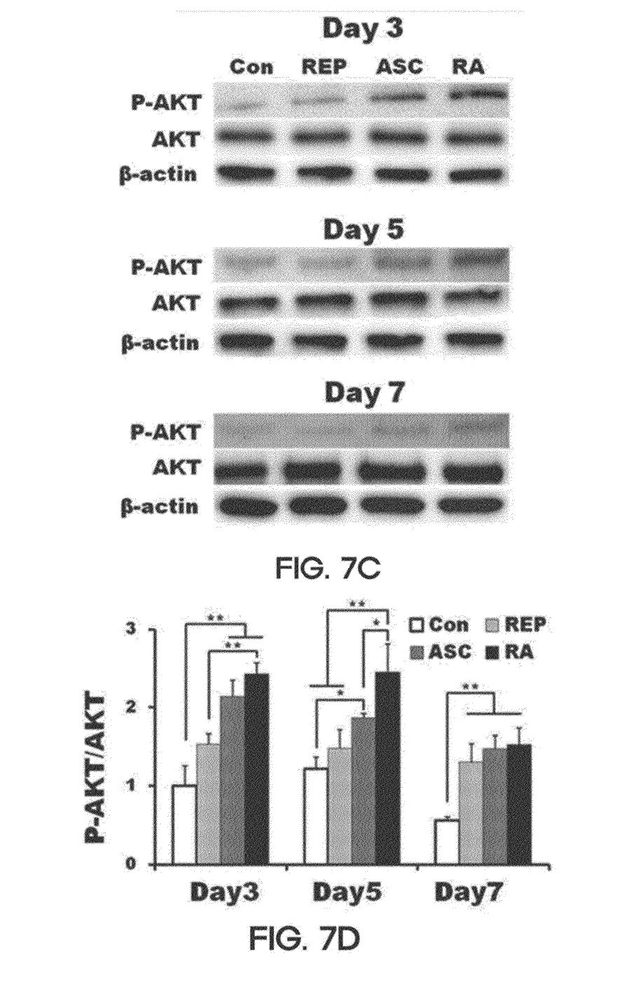

Finally, FIG. 7 confirms the increase of Erk and Akt phosphorylation through REP during wound-healing, and it was confirmed that the ratios of p-ErK/Erk and p-Akt/Akt in the group treated with the combination of REP and ASC significantly increased compared with other groups. That is, skin wounds can be more effectively treated by simultaneously applying both REP and ASC on the wounds to induce the phosphorylation of Erk and Akt in ASC by REP, and thereby promote angiogenesis.

The present invention provides a method for wound-healing or promotion of wound-healing by using the composition for wound-healing or promotion of wound-healing.

The composition for wound-healing or promotion of wound-healing of the present invention may be prepared according to a method known in the pharmaceutical field, and may be prepared in various formulations such as the conventional pharmaceutical formulations, e.g., liquids, ointments, emulsions, gels, creams, pastes, etc., by mixing with the construct or a pharmaceutically acceptable carrier or excipient. The preferable dose of the therapeutic agent for cell regeneration of the present invention, although not particularly limited, may vary depending on the health state, body weight of a patient, severity of the disease(s) and the symptoms, drug type, and duration, but may be appropriately selected by those skilled in the art. For preferable effect, the therapeutic agent may be conventionally administered at a concentration of 25 μM to 100 μM daily per each wound, and preferably, 45 μM to 65 μM. The administration may be performed once daily or divided into several doses for a day.

Hereinafter, the present invention will be described in more detail with reference to Examples. However, it should be obvious to those skilled in the art that these Examples are for illustrative purposes only, and the invention is not intended to be limited by these Examples.

### Example 1

### Preparation of Multiblock Biopolymer (REP) and Confirmation of its Characteristics

The purification of REP and confirmation of particular transition temperature (T) were prepared in the same manner as described in the journal (Stimulation of fibroblasts and neuroblasts on a biomimetic extracellular matrix consisting of tandem repeats of the elastic VGVPG domain and RGD motif (Jeon W B et al., J. Biomed. Mater. Res. A. 97:152, 2011).

For the conjugation of 5-carboxyfluorescein (Fam) to the N-terminus of REP, 5-carboxyfluorescein N-succinimidyl ester (Sigma, USA) was dissolved in 580 μL of DMSO to a concentration of 5 μmol, and then added with 20 mL of PBS containing 0.97 mol REP. The mixture was reacted at room temperature for 3 hours and thereby prepared a Fam-labeled REP (Fam-REP). The Fam-REP was purified by inverse phase transition. The level of labeling was measured according to the protocol included in the AnaTag™ protein labeling kit (AnaSpec, USA).

In the presence of DTT, the level of inverse phase transition by REP was measured at REP concentrations (20 μM, 50 μM, and 100 μM) and according to temperature change. The temperature was allowed to increase at the rate of 1° C./min. As a result, it was observed that the absorbance rapidly increased at 25° C. or higher (FIG. 1A), and the REP agglutination at 35° C. in the state of coacervates were measured according to the concentration (FIG. 1B).

Additionally, when the level of inverse phase transition by Fam-REP was measured in the presence of DTT, the absorbance rapidly increased at 30° C. or higher (FIG. 1C). The change in absorbance was measured using UV-visible spectrum according to Fam-REP wavelength (FIG. 1D), and a peak was shown to appear at about 500 nm.

### Example 2

### Isolation of Adipose Stem Cells and Confirmation of their Characteristics

Enhanced green fluorescent protein (EGFP)-labeled adipose stem cells (hereinafter, “ASC”) were isolated from C57BL/6-GFP mice (Park J K et al., et al., Cell Transplant, 21:2407, 2012), and the characteristics of ASC were analyzed by flow cytometry.

ASC was cultured in a medium under the conditions of 37° C., 5% CO₂. When the culture container was filled about 70%, it was treated with trypsin and subjected to subcultures. After performing a total of four subcultures, the ASC therefrom was used in the experiments.

Approximately 5×10⁵ cells were washed twice with PBS and cultured after adding with phycoerythrin (PE)-conjugated rat anti-mouse CD31, CD34, CD45, CD13, CD29, CD44, and CD90 antibodies. PE-rat IgG1 was used as a control, and all antibodies used were purchased (BD science, USA).

As a result, the ASC isolated in the present invention showed a cluster of differentiation markers for CD13, CD29, CD44, and CD90 as positive, and CD31, CD34, and CD45 were observed to be negative (FIG. 2).

### Example 3

### Confirmation of Wound-Healing Effect by Treatment with ASC and REP

3-1: Preparation of Animals

Eight week-old male C57BL/6 mice (20 g to 30 g), which are specific pathogen free (SPF), were purchased (Central Lab. Animal Inc., Korea). The C57BL/6 mice have been numerously used as experimental animals in studies on skin injuries, and they may be used as references in the application of other experimental results.

The animals were bred in an animal facility under the controlled conditions of 22±3° C., 50±10% of relative humidity, and lighting for 12 hours followed by 12 hours of darkness. The experimental mice were accommodated one per each polycarbonate breeding box, and the animals were given ad libitum access to solid feeds for experimental animals (PMI Nutritional International, Richmond, USA) after sterilizing them using UV irradiation (13.2 kGy) and also ad libitum access to filtered tap water using bottles. All the management and surgery of the experimental animals were approved by the Animal Experimentation Ethics Committee of Daegu Gyeongbuk Institute of Science and Technology (DGIST).

3-2: Formation of Cut-Wound and Treatment of the Wound with ASC and REP

In order to examine the effect of simultaneous administration of ASC and REP, the experimental mice were divided into a Sham control, a REP-treated group, an ASC-treated group, and an ASC-REP combined treatment group (RA), and a quantitative analysis on the wound-healing was performed. The experimental mice were arbitrarily divided into the four groups (15 mice/group), and the cut-wounds were generated on the dorsal region of the mice to a size of 8 mm in diameter using a round biopsy punch. Then, each group was treated on the wounds as follows: 50 μL of PBS (Sham control), 50 μM REP (REP-treated group), 1×10⁶ ASC (ASC-treated group), or a combination of 1×10⁶ ASC and 50 μM REP (ASC-REP combined treatment group; hereinafter “RA group”).

Over the entire experimental period, the level of shrinkage of the cut-wound on the skin was observed by the naked eye. The wounds were covered with Tegaderm (3M Health Care, USA) for 7 days to prevent a secondary infection and maintained not to be dry. The mice showed no disease due to the external skin wounds. Additionally, for comparison with the initial cut-wound tissues, the tissues on the 0^{th} day (after generation of the cut-wound) were isolated after making the size of the wound to have a diameter of 10 mm using a round biopsy punch.

3-3: Measurement of the Level of Wound Closure

On the 0^{th}, 3^{rd}, 5^{th}, 7^{th}, and 14^{th} day after the generation of cut-wounds, the relative area and the rate of wound closure in each cut-wound (the lower the result value the higher the rate of wound closure) were measured.

The area of wound was measured by Equation 1 below, and the rate of wound closure (%) was measured by Equation 2 below.

longest length×shortest length×π [Equation 1]

(area of wound by time for REP treatment/area of wound on the 0^{th} day by REP treatment)×100 [Equation 2]

For the histological analysis, tissue samples were obtained, fixed in 10% neutral buffered formalin solution, embedded using paraffin wax, and sliced to have a thickness of 4 μm. H&E staining and Masson's trichrome (MT) staining were performed according to the known method (Park J K et al., et al., Cell Transplant, 21:2407,2012), and histological images were obtained using Leica microscope equipped with ProgRes® CapturePro software (version 2.8.8, Germany). The areas of micrographic granulation tissues and collagen deposition were measured using an image analysis system (IMT i-Solution, Inc., Canada). Re-epithelialization was measured in percentage relative to the initial wound area by surgery (Malinda K. M. et al., Int. J. Biochem. Cell Biol., 40:2771, 2008; Lemo N. et al., Vet. Arh., 80:637, 2010).

As a result, as shown in FIGS. 3A and 3B, it was confirmed that all the wounds in the REP-treated group, the ASC-treated group, and the ASC-REP combined treatment group were closed in all wound-healing steps, compared with that of the control group (Sham Control), and the rate of wound closure relatively increased in the order of REP, ASC, and RA (REP+ASC). Additionally, in line with the above result, high re-epithelialization was observed in the RA group compared with other groups (FIGS. 3C and 3D).

3-4: Confirmation of α-SMA Expression

α-SMA (Alpha-smooth muscle actin) is known as a myofibroblast-forming marker, and in the present invention, the level of α-SMA expression in each group was measured by Western blot analysis.

The tissue proteins were isolated according to the manual using RIPA buffer (Sigma) and Halt Phosphatase Inhibitor Cocktail (Thermo, USA).

Western blot analysis was performed referring to the previous study (Lee K. M. et al. Acta. Biomater., 9:5600, 2013). Anti α-SMA (anti-α-SMA) antibodies were purchased from Cell Signaling Technology (USA) using β-actin as a control. Western blot analysis was performed using Gel Logic 4000 Pro Imaging System (Carestream, USA), and the quantitative analysis of band density was performed using Molecular Imaging Software (Carestream, USA).

As a result, as shown in FIGS. 3E and 3F, the RA group showed a 1.4-, 1.4-, and 1.2-fold increase in α-SMA expression level on the 3^{rd}, 5^{th}, and 7^{th} day of the experiment, compared with the ASC treatment alone, respectively.

### Example 4

### Measurement of Restoration of Local Vascular Structure by ASC and REP Treatment

4-1: Measurement of Expression Amount of VEGF, CD31, and VWF

In order to examine the angiogenesis and vascularity in tissue regeneration by ASC, the expression levels of vascular endothelial growth factor (VEGF), CD31, and Von Willebrand Factor (VWF) according to each group in Example 3 were measured by enzyme-linked inmmunosorbent assay, enzyme-linked immunospecific assay (ELISA).

The ELISA kit for the measurement of VEGF, CD31, and VWF were purchased from MyBioSource (USA), and the protein content in tissues were measured according to the Manufacturer's manual.

As a result, as shown in FIG. 4A, the content of VEGF in REP, ASC, and RA groups showed an increase of 126%, 132%, and 147% for two weeks compared with that of the control group, respectively. The VEGF content in REP, ASC, and RA groups started to increase from the 3^{rd} day, reached the highest level on the 5^{th} and 7^{th} day, and decreased on the 14^{th} day. In contrast, the control group (negative control group) showed a slow increase from the 1^{st} day and reached the highest on the 14^{th} day.

Additionally, as shown in FIG. 4B, the total content of CD31 in the REP, ASC, and RA groups showed an increase of 137%, 226%, and 271% for two weeks compared with that of the control group, respectively. In all experimental groups, the CD31 content showed a gradual increase, reached the highest on the 7^{th} day, and decreased on the 14^{th} day.

As shown in FIG. 4C, the amount of VWF production increased along with the progress of wound-healing, and the largest amount of VWF was accumulated in the wound treated with RA. The relative amount of VWF measured in control group, REP, ASC, and RA groups for two weeks were confirmed to be 100%, 115%, 150%, and 182%, respectively.

4-2: Immunofluorescent Analysis

The amount of CD31 expression in wound tissues treated with REP and/or ASC in each group of Example 3 was analyzed via immunofluorescent analysis.

In order to examine the CD31 expression in tissues treated with REP and/or ASC, rabbit anti-CD31 antibody (Abcam, England) and Alexa Fluor 568-conjugated anti-mouse IgG (Invitrogen, USA) were used. The observation of cells were performed using 4′,6′-diamidine-2′-phenylindole dihydrochloride (DAPI) staining method, and the analysis was performed using a known method (Alexaki V I., et al., Cell Transplant, 21:2441, 2012). The fluorescent microscopic images were obtained using Leica DMI 3000 fluorescent microscope and LSM 700 confocal microscope (Carl Zeiss).

As a result, it was confirmed that the amount of CD31 expression was highest in RA group, and this agreed with the result of Example 4-1. That is, in the group which was treated with REP and ASC simultaneously, the formation of microvascular structure was promoted in all steps (FIG. 4D).

Additionally, as shown in Example 2, the ASC used in the present invention was in a EGFP-labeled state. Therefore, as a result of the simultaneous measurement of the expression amount of EGFP and CD 31, as shown in FIG. 4E, when the wound was treated with ACS alone, the positive cells expressing both CD31 and EGFP could not be detected, whereas when the wound was treated with REP and ASC simultaneously, the cells expressing CD31 and EGFP simultaneously along the cross section of tubular structure were confirmed.

That is, the discovery of the cells showing positive in CD31 and EGFP along with the cross section of newly produced vascular tubules indicate that ASC is a phenotype of an endothelial cells and is directly involved in the differentiation and regeneration of newly developed vessels.

### Example 5

### Effect of REP on Increase of ASC Viability

In order to examine whether REP has a direct effect on the increase of viability rate of the transplanted ASC, the expression level of EGFP being shown in ASC was observed under fluorescent microscope.

As a result, as shown in FIG. 5A, the ASC where EGFP is expressed (hereinafter “EGFP-ASC”) was detected in greater amount in the RA group than in the group treated with ASC alone. Along with the progress of wound-healing, the number of EGFP-ASC was decreased relatively in both groups and almost no EGFP-ASC was observed on the 7^{th} day. On the 5^{th} day, the relative size of ASC observed in the group treated with ASC alone was significantly smaller than the cells observed in the RA group. On the 7^{th} day, the ASC in the RA group lost the typical long spin shape and became more round, and this indicates that the ASC was being differentiated into different cells or reached close to apoptotic cell death.

Additionally, the cell number of ASC was relatively measured by analyzing the expression level of EGFP, which is expressed in ASC by Western blot analysis. The isolation of cellular proteins and Western blot analysis were performed in the same manner as in Example 3-4, and the antibodies for performing Western blot analysis and the antibodies that specifically recognize EGFP were purchased from Cell Signaling Technology (USA) and measured using β-actin as a control.

As a result, as shown in FIGS. 5B and 5C, on the 1^{st} day after ASC and/or REP transplantation, it was confirmed that the remaining transplanted ASC (1×10⁶ cells) were those corresponding to 40% and 55% in the group treated with ASC alone and in the RA group, respectively. Additionally, it was confirmed that 11% and 19% were remaining on the 3^{rd} day and 6.7% and 8.1% on the 5^{th} day in each group, respectively. Comparing with the wound treated with ASC alone, the combined treatment of REP and ASC showed an increase in cell viability by about 24%, 40%, 17%, and 35% on the 1^{st}, 3^{rd}, 5^{th}, and 7^{th} day, respectively.

### Example 6

### Confirmation of ASC Adhesion Capacity in REP and Confirmation of Activity of Fak, Src, Erk and Akt Phosphorylation

6-1: Confirmation of ASC Adhesion Capacity in REP

The adhesion level of ASC was compared in REP, collagen I, collagen IV and fibronectin. Using a general cell culture container without any treatment as a control, a culture container coated with collagen I, collagen IV, and fibronectin, and REP were treated with ASC (1×10⁶ cells) isolated in Example 2, and the level of adhesion was measured according to time. The ASC adhered to REP, collagen I, collagen IV, and fibronectin were stained by Crystal violet staining method and observed under microscope, and the level of cell adhesion was confirmed by measuring absorbance at 570 nm.

As a result, as shown in FIGS. 6A and 6B, 30 minutes after the culture, the highest cell adhesion rate was shown in fibronectin, followed by collagen I and collagen IV in this order. After one hour of culture, the number of cells adhered to REP was observed to be similar those in collagen I and collagen IV, and about 70% of cells were shown to be adhered in fibronectin. After two hours of culture. REP, collagen I, collagen IV, and fibronectin showed a similar level of cell adhesion, however, after three hours of culture, the number of cells adhered in fibronection was confirmed to be highest.

6-2: Confirmation of Activity of Fak, Src, Erk, and Akt Phosphorylation

The level of activity of Fak. Src, Erk, and Akt phosphorylation was confirmed by culturing while allowing adhesion of ASC cells to REP, collagen I, collagen IV, and fibronectin.

The level of Fak, Src, Erk, and Akt phosphorylation was measured by Western blot analysis, and the cells were cultured in each scaffold and separated after 30 minutes and subjected to Western blot analysis in the same manner as in Example 3-4. The antibodies for Fak, p-Fak, Src, p-Src, Erk, p-Erk, Akt, and p-Akt were purchased from Cell Signaling Technology (USA) and measured using β-actin as a control.

As a result, the p-Fak/Fak ratio was shown to be at similar levels in REP and collagen I, and the phosphorylation level of Fak was increased about twice compared with those of collagen IV and fibronectin (FIGS. 6C and 6G). Additionally, the phosphorylation level of Src was shown to be very similar to that of Fak (FIGS. 6D and 6H), and the phosphorylation level of ErK was measured to be higher than other scaffolds in REP (FIGS. 6E and 6I). In the ease of Akt, the p-Akt/Akt ratio was observed to be lowest in collagen I, whereas it was shown to be at similar levels in REP, collagen I, collagen IV, and fibronectin (FIGS. 6F and 6J).

Analyzing the above results, although fibronectin was observed to have excellent cell adhesion rate than REP, however, the induction of activity of Fak, Src, Erk, and Akt (phosphorylation) was shown to be significantly increased compared with other scaffolds when ASC was cultured in REP.

That is, REP not only serves to induce the phosphorylation of Erk and Akt in ASC cells thereby promoting angiogenesis but also increases the activity of Fak and Src thereby contributing to the migration of endothelial cells to the wound.

### Example 7

### Increase of Erk and Akt Phosphorylation by REP During Wound-Healing

Based on the result of Example 6, in order to examine the level of increase in Erk and Akt phosphorylation of ASC by REP during the actual wound-healing process, the cells were collected from each experimental group in Example 3, and Western blot analysis was performed in the same manner as in Example 3-4 or Example 6.

As a result, the RA group showed the highest p-ErK/Erk ratio in both ASC group and REP group on the 3^{rd}, 5^{th}, and 7^{th} day of the wound-healing process (FIGS. 7A and 7B). The control group induced the lowest Erk phosphorylation, and Erk phosphorylation was shown high on the on the 3^{rd} and 5^{th} day in all groups. On the 7^{th} day, the Erk phosphorylation was decreased, and the level of relative decrease compared with that of the 5^{th} day in control, REP, ASC, and RA groups were shown to be 48%, 39%, 39%, 23.3%, respectively.

Akt phosphorylation showed the highest p-Akt/Akt ratio in the RA group compared with other groups (FIGS. 7C and 7D) and showed a similar expression pattern to that of Erk. On the 7^{th} day, the level of decrease in Akt phosphorylation compared with that of the 5^{th} day, the relative level of decrease in control, REP, ASC, and RA groups were shown to be 54%, 12%, 21%, and 38%, respectively.

That is, the simultaneous treatment of REP and ASC on wounds can induce the phosphorylation of Erk and Akt in ASC cells by REP to thereby promote angiogenesis and more effectively treating skin wounds.

The invention has been described in detail with reference to preferred embodiments thereof. However, it will be appreciated by those skilled in the art that the specific technical features are merely preferred embodiments of the present invention and they should not be construed as limiting the scope of the present invention, and thus the substantial scope of the present invention shall be defined in the appended claims and their equivalents.

What is claimed is:

1. A method for wound-healing or promoting wound-healing, comprising administering to a wound of a subject an adult stem cell and a multiblock biopolymer (REP) established by a repeated fusion between an elastin-like polypeptide; and a ligand.

2. The method of claim 1, wherein the adult stem cell is at least one kind of a mesenchymal stem cell, a neural stem cell, or a hematopoietic stem cell selected from the group consisting of an adipose-derived stem cell, a bone marrow-derived stem cell, and an umbilical cord-derived stem cell.

3. The method of claim 1, wherein the elastin-like polypeptide is an elastin VGVPG (valine-glycine-valine-proline-glycine) peptide (polypeptide).

4. The method of claim 1, wherein the ligand is RGD (arginine-glycine-aspartate) or RGDS (arginine-glycine-aspartate-serine).

5. The method of claim 1, wherein the multiblock biopolymer is [VGRGD(VGVPG)₆]ₙ (wherein n=10, 12, 15, or 20).

6. The method of claim 1, comprising 25 μM to 100 μM of a multiblock biopolymer and 5×10⁵ to 5×10⁶ adult stem cells.


SEQUENCE LISTING (5 sequences)

SEQ ID NO: 1 - protein, 5 aa, Artificial
  Elastin-like polypeptide
VGVPG

SEQ ID NO: 2 - protein, 3 aa, Artificial
  Ligand
RGD

SEQ ID NO: 3 - protein, 4 aa, Artificial
  Ligand
RGDS

SEQ ID NO: 4 - protein, 17 aa, Artificial
  Multiblock biopolymer(REP)
TGPGVGRGDVGVPGWPC

SEQ ID NO: 5 - protein, 10 aa, Artificial
  Multiblock biopolymer(REP)
VGRGDVGVPG